(19) United States
(12) Patent Application Publication
(10) Pub. No.: US 2020/0323213 A1
(43) Pub. Date: Oct. 15, 2020

(54) Methods of use for compositions comprising biguanide compound and diol surfactant

(71) Applicant: 3M INNOVATIVE PROPERTIES COMPANY, St. Paul, MN (US)

(72) Inventors: Petra L. Kohler Riedi, Minneapolis, MN (US); Jimmie R. Baran, Jr., Prescott, WI (US)

(21) Appl. No.: 16/911,420

(22) Filed: Jun. 25, 2020

Related U.S. Application Data
(62) Division of application No. 15/518,568, filed on Apr. 12, 2017.
(60) Provisional application No. 62/081,182, filed on Nov. 18, 2014.

(51) Int. Cl. A01N 47/44; A61K 8/43; A61K 31/155; A61K 31/785; A61K 31/047; A01N 25/30; A01N 31/02

(52) U.S. Cl. CPC A01N 47/44; A61K 8/43; A61K 31/155; A01N 31/02; A61K 31/047; A01N 25/30; A61K 31/785

(57) ABSTRACT

Method of use for antimicrobial compositions are described comprising a least one biguanide compound and at least one hydrocarbon surfactant comprising a saturated or unsaturated C₂-C₄ diol moiety and optionally further comprising alkoxy groups. In some embodiments, the composition further comprises an aqueous or hydrophilic liquid carrier. In favored embodiments, the diol surfactant is present in an amount such that the antimicrobial activity of the biguanide antimicrobial compound is improved. Also described are articles comprising the described antimicrobial composition.

### CROSS REFERENCE TO RELATED APPLICATIONS

This application is a divisional application of U.S. application Ser. No. 15/518,568, filed Apr. 12, 2017, which is a national stage filing under 35 U.S.C. 371 of PCT/US2015/060549, filed Nov. 13, 2015, which claims the benefit of U.S. Provisional Application No. 62/081,182, filed Nov. 18, 2014, the disclosure of which is incorporated by reference in its/their entirety herein.

### SUMMARY

Presently described is an antimicrobial composition comprising a least one biguanide compound and at least one hydrocarbon surfactant comprising a saturated or unsaturated C₂-C₄ diol moiety, optionally further comprising alkoxy groups. In some embodiments, the composition further comprises an aqueous or hydrophilic liquid carrier.

The surfactant typically has the formula

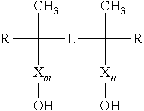

wherein L is a covalent bond or a saturated or unsaturated C₁-C₂ moiety, R is independently a straight chained or branched C₁-C₁₂ alkyl moiety, X is independently a C₂-C₄ alkoxy, and m+n ranges from 0 to about 50.

In favored embodiments, the diol surfactant is present in an amount such that the antimicrobial activity of the biguanide antimicrobial compound is improved.

Also described are articles comprising the described antimicrobial composition. In some embodiments, the composition is absorbed or disposed on a carrier substrate.

Also described are methods of use comprising providing the described antimicrobial composition and applying the composition to a surface.

### DETAILED DESCRIPTION

The compositions described herein comprise an antimicrobial. “Antimicrobial” means a chemical agent other than an “enhancer” as described herein that kills pathogenic and non-pathogenic microorganisms. When utilized to kill or prevent the growth of microorganism on a mammal, such as humans, the term antimicrobial can be synonymous with the term antiseptic. Antiseptics generally interfere more broadly with the cellular metabolism and/or the cell envelope.

The antimicrobial composition comprises an effective amount of one or more biguanide compounds. The biguanide compound is generally considered the main active component of the composition. As used herein the term biguanide includes bisbiguanides.

In some embodiments, the biguanide compound can be represented by the formula:

R-NHC(NH)NHC(NH)NH(CH₂)ₙNHC(NH)NHC(NH)NH-R

where n=3-10, preferably 4-8, and most preferably 6; and R is a C₄-C₁₈ branched or straight chain alkyl, C₆-C₁₂ aryl or alkaryl, each of which may be optionally substituted in available positions by halogen. In some embodiments, the molecular weight of such biguanide compound is no greater than 1000, 900, 800, 700, or 600 g/mole.

One preferred compound of this class is chlorhexidine. This may be present as the free base but is typically present as a disalt of acetate, gluconate, lactate, methosulfate (CH₃OSO₃⁻), or a halide or combinations thereof. Diacetate, digluconate, dilactate, and dimethosulfate salts can be preferred since these salts exhibit a solubility in aqueous fluid in excess of 1 g/100 ml. For example, the solubility of the digluconate salt is 20 g/100 ml and that of the diacetate is 1.9 g/100 ml. The most common compound is chlorhexidine digluconate (CHG). Other anions may be useful. The counter ion typically ensures sufficient solubility in aqueous fluid.

In one embodiment, the biguanide compound can be characterized as a polybiguanide. Compounds of this class can be represented by the formula:

X-R¹-[NHC(NH)NHC(NH)NH-R²]ₙ-X

where R¹ and R² are bridging groups such as alkylene groups typically having 2 to 10 carbons atoms, 4 to 8 carbons atom, and most typically 6 carbon atoms. The alkylene bridging groups can be optionally substituted in available positions with halogen, hydroxyl, or phenyl groups. X is a terminal group and is typically an amine, amine salt, or a dicyandiamide group. The number of repeat units, “n” can range from 2 to about 50. In some embodiments, the average molecular weight (Mw) of such biguanide compounds can range up to 10,000 g/mole. In other embodiments, the average molecular weight (Mw) of such biguanide compounds is no greater than 5,000 or 1,000 g/mole. A preferred compound of this class is polyhexamethylene biguanide (PHMB) commercially available as Cosmocil CQ from Lonza Chemicals, Allendale, N.J.

One or more biguanide compounds are present in the composition in an effective amount to provide the desired antimicrobial properties.

In typical embodiments, the composition comprises at least one antimicrobial biguanide compound and at least one saturated or unsaturated (e.g. acetylenic) C₂-C₄ diol surfactant dissolved or dispersed in an aqueous liquid carrier. By “aqueous” it is meant that the liquid carrier comprises at least 50, 60, 70, 80 wt-% to 100 wt-% water.

In other embodiments, the composition comprises at least one antimicrobial biguanide compound and at least one saturated or unsaturated (e.g. acetylenic) C₂-C₄ diol surfactant dissolved or dispersed in a hydrophilic liquid carrier. By “hydrophilic liquid carrier” it is meant that the liquid carrier comprises greater than 50, 60, 70, 80 wt-% of a liquid carrier other than water. In some embodiments, the water content is less than 20, 15, 10 or 5 wt-%. In typical embodiments, the water (when present) and hydrophilic liquid are miscible at 23° C. forming a stable continuous liquid phase.

The hydrophilic liquid carrier may comprise liquids which may be considered “an additional enhancer” provided that the inclusion of such does not mask the enhancing effect of the saturated or unsaturated (e.g. acetylenic) C₂-C₄ diol surfactant. For example, if the combination of biguanide antimicrobial and other enhancer has a greater efficacy than the combination of biguanide and saturated or unsaturated (e.g. acetylenic) C₂-C₄ diol surfactant, the enhancing effect of the diol surfactant is typically not detectable. The enhancer component may include an alpha-hydroxy acid, a beta-hydroxy acid, other carboxylic acids, a (C₁-C₄)alkyl carboxylic acid, a (C₆-C₁₂)aryl carboxylic acid, a (C6-C12)aralkyl carboxylic acid, a (C6-C16)alkaryl carboxylic acid, a chelator, a phenolic compound (such as certain antioxidants and parabens), a (C1-C10)monohydroxy alcohol, or a glycol ether (i.e., ether glycol). Various combinations of enhancers can be used if desired.

In some embodiments, the hydrophilic liquid carrier comprises a monohydroxy alcohol having 1-10 carbon atoms. This includes the lower (i.e., C1-C4) monohydroxy alcohols (e.g., methanol, ethanol, isopropanol, and butanol) as well as longer chain (i.e., C5-C10) monohydroxy alcohols (e.g., isobutanol, t-butanol, octanol, and decanol). In certain embodiments, the alcohols useful in the compositions of the present invention are selected from the group consisting of methanol, ethanol, isopropyl alcohol, and mixtures thereof.

In some embodiments, at least one (e.g. short chain C1-C4) alcohol is present in a total amount of at least 0.5, 1, 2, 3, 4 or 5 wt-%. In some embodiments, the concentration of alcohol is no greater than 50, 40, 30, or 20 wt-% of the ready to use composition.

For certain applications, lower alcohols may not be preferred due to the strong odor and potential for stinging and irritation. This can occur especially at higher levels. In applications where stinging or burning is a concern, the concentration of (C1-C4) alcohols is preferably less than 20%, more preferably less than about 15%.

In one embodiment, longer chain (i.e., C5-C10) alcohols are present in a total amount of at least 0.1, 0.25, 0.5 wt-% or 1.0%, based on the ready to use composition. The (C5-C10) alcohols are typically present in a total amount of no greater than 10, 5, 4, 3, or 2 wt-%, based on the total weight of the ready to use composition.

In another embodiment, the hydrophilic liquid comprises an ether glycol. Exemplary ether glycols include those of the formula:

R′—O—(CH₂CHR″O)ₙ(CH₂CHR″O)H

wherein R′ is H, a (C1-C8) alkyl, a (C6-C12) aryl or a (C6-C12) aralkyl or (C6-C12) alkaryl; and each R″ is independently=H, methyl, or ethyl; and n=0-5, preferably 1-3. Examples include 2 phenoxyethanol, dipropylene glycol, triethylene glycol, the line of products available under the trade designation DOWANOL DB (di(ethylene glycol) butyl ether), DOWANOL DPM (di(propylene glycol)monomethyl ether), and DOWANOL TPnB (tri(propylene glycol) monobutyl ether), as well as many others available from Dow Chemical, Midland Mich.

In one embodiment, ether glycols are present in a total amount of at least 0.1, 0.25, 0.5 wt-% or 1.0%, based on the ready to use composition. The ether glycols are typically present in a total amount of no greater than 20, 15, 10, 5, 4, 3, or 2 wt-%, based on the total weight of the ready to use composition.

In yet another embodiment, the composition comprising at least one biguanide antimicrobial compound and at least one (e.g. acetylenic) diol surfactant typically but optionally diluted in a liquid medium, such as water, can be applied to a carrier substrate such as nonwoven and dried. In this embodiment, the (i.e. dried) composition is disposed on the carrier substrate. The enhanced antimicrobial can be reactivated by applying an aqueous or hydrophilic liquid to the dried carrier substrate.

The effective amount of biguanide antimicrobial compound is typically no greater than about 5, 4, 3, 2, or 1 wt-% based on the total weight of the aqueous or hydrophilic composition. The effective amount is more typically no greater than 0.9, 0.8, 0.7, 0.6, 0.5, 0.4, 0.3, 0.2 or 0.1 wt.-% based on the total weight of the composition. In some embodiments, the effective amount may be as little as 0.09, 0.08, 0.07, 0.06, 0.05, 0.04, 0.03, 0.02 or 0.01 wt.-%. In some embodiments, the effective amount may be as little as 0.009, 0.008, 0.007, 0.006, 0.005, 0.004, 0.003, 0.002 or 0.001 wt-%. The composition described herein may however be a concentrated composition that is typically diluted prior to use. Such concentrated compositions may comprise as much as 10 wt-% or greater of biguanide compounds(s).

It will be appreciated by one of ordinary skill in the art that the levels or ranges selected for the required or optional components described herein will depend upon factors such as whether one is formulating a composition for direct use, or a concentrate for dilution prior.

The compositions described here further comprise at least one surfactant compound based on a saturated or unsaturated (e.g. acetylenic) diol or a derivative thereof. The saturated or unsaturated (e.g. acetylenic) diol surfactant compound functions as an enhancer for the antimicrobial. “Enhancer” means a component that enhances the effectiveness of the antimicrobial component. The composition comprising both the antimicrobial component and the enhancer component provides improved antimicrobial activity relative to these components being utilized separately. The enhancing effect can be with respect to the level of kill, the speed of kill, and/or the spectrum of microorganisms killed. The enhancing effect may be evident with respect to some microorganisms and absent with respect to others. The enhancer effect is typically synergistic such that the composition as a whole displays an activity that is greater than the sum of the activity of the antimicrobial compound alone and the enhancer compound alone.

In some embodiments, the surfactant includes those according to the following general structure:

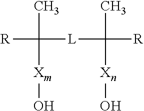

wherein L is a covalent bond or a saturated or unsaturated C₁-C₂ moiety, R is independently a straight chained or branched C₁-C₁₂ alkyl moiety, X is independently a C₂-C₄ alkoxy, and m+n ranges from 0 to about 50.

In some embodiments, the unsaturated moiety comprises a double bond or in other words L is an alkene moiety. In other embodiments, the unsaturated moiety comprises a triple bond and L is an alkyne moiety.

In some embodiments, the acetylenic diol compounds include those according to the following general structure:

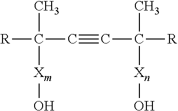

wherein R is independently a C₁-C₁₂ alkyl which may be straight chained or branched; X is independently selected from C₂-C₄ alkoxy (e.g. ethoxy, propoxy and butoxy); m+n is a number of moles of C₂-C₄ alkoxy and ranges from 0 to about 50.

In both of these structures preferably, R is a straight chained or branched C₁-C₆ alkyl, X is an ethoxy moiety, and m+n is from 0 to about 30. In some embodiments, m+n is 0. In other embodiments, m+n is at least 1, 2, or 3. In some embodiments, m+n is no greater than 20, 15, or 10.

Various acetylenic diol surfactants are commercially available under the trade designation SURFYNOL (ex. Air Products Inc., Allentown, Pa.) Non-limiting examples include SURFYNOL 104 which is described as 2,4,7,9-tetramethyl-5-decyn-4,7-diol (also sometimes referred to as “tetramethyl decynediol”) (wherein m+n equals 0); as well as SURFYNOL 82, described to be dimethyl octynediol (R is CH₂CH₃).

Other useful acetylenic diol compounds are ethoxylated derivates. In this embodiment, m, n and more typically are m+n is a number of moles of ethoxy.

Various ethoxylated derivatives of acetylenic diol surfactants are commercially available under the trade under the trade designation SURFYNOL (ex. Air Products Inc., Allentown, Pa.) Non-limiting examples include SURFYNOL 440, SURFYNOL 465 and SURFYNOL 485. More specifically, SURFYNOL 465 is described as being the reaction product of approximately 10 moles of ethylene oxide (m+n equals 10) with 1 mole of tetramethyl decynediol. SURFYNOL 485 is described to be the reaction product of approximately 30 moles of ethylene oxide such that each X is ethoxy and (m+n equals 30) with 1 mole of tetramethyl decynediol. Other ethoxylated acetylenic diols are available from Air Products under the tradename DYNOL (e.g., DYNOL 604 and DYNOL 607).

A suitable alkane diol is 2,4,7,9-tetramethyldecane-4,7-diol available from Air Products as “SURFYNOL AD01 SURFACTANT”. The total concentration by weight of antimicrobial biguanide compound(s) relative to the total concentration of saturated or unsaturated (e.g. acetylenic) diol surfactant(s) by weight can generally ranges from about 100:1 to about 1:20. In some embodiments, the total concentration by weight of biguanide antimicrobial compound(s) relative to the total concentration of saturated or unsaturated (e.g. acetylenic) diol surfactant(s) by weight is no greater than 75:1, 50:1, 25:1, 10:1, or 4:1. The weight ratios are applicable for aqueous and hydrophilic compositions, as well as for embodiments wherein the antimicrobial biguanide compound(s) together with the saturated or unsaturated (e.g. acetylenic) diol surfactant(s) are present on a surface after removal (by evaporation or active drying) of the aqueous or hydrophilic liquid medium.

In another embodiment, the composition may be a thickened aqueous gel or a thickened hydrophilic gel prepared by adding a thickener to the aqueous or hydrophilic liquid carrier.

The thickener system can include (e.g. soluble or swellable) organic polymers or inorganic thixotropes such as silica gel, clays (such as betonite, hectorite, montmorrillonite and the like), as well as organically modified inorganic particulates materials, and the like.

The total concentration of the thickener system is typically less than 10, 9, 8, 7, 5, 4, or 3 wt-% based on the total weight of the ready to use composition. The total concentration of the thickener is typically at least 0.5 wt-% or 1 wt-%, based on the total weight of the ready to use composition.

Biguanides, such as chlorhexidine and PHMB, are very basic and capable of forming multiple ionic bonds with anionic materials. For this reason, biguanide-containing compositions are generally free of anioinic compounds that can result in precipitation of the antimicrobial. For this reason, thickener systems, if present, are typically based on non-ionic and/or cationic polymers. Further, if additional surfactants are present such surfactants are typically zwitterionic, very water soluble, non-precipitating anionic emulsifiers, or non-ionic emulsifiers such as poloxamers. If a salt is further included for stability or other purposes, preferably gluconate salts such as triethanolamine gluconate or sodium gluconate, are used.

The composition is generally free of anionic components such as anionic surfactants and/or additional anionic antimicrobials, as well as halide salts. As used herein, “free of” a particular constituent means that the composition contains less than 0.0001% wt. of the particular constituent, and preferably the composition is essentially free of the particular constituent.

Cationic polymeric thickeners include for example cationically modified celluloses, quaternized natural amino-functional polymers, and polymers based on ethylenically unsaturated monomers selected from the group consisting of acrylates, acrylamides, vinyl lactams, vinyl acetates, methyl vinyl ethers, styrene, and acrylonitrile.

Nonionic polymeric thickeners include for example modified celluloses, guar, xanthan gum, and other natural polymers such as polysaccharides and proteins, associative polymers based on nonionic ethylenically unsaturated monomers wherein at least one comonomer has at least 16 carbon atoms, and polymers based on ethylenically unsaturated monomers selected from the group consisting of acrylates, acrylamides, vinyl lactams, vinyl acetate and its hydrolyzed derivatives, methyl vinyl ethers, styrene, and acrylonitrile.

A variety of cellulosic ethers are reported in the literature to be soluble in water. Materials in this class that are nonionic and have been shown to be useful include: methylhydroxypropylcellulose, available as BENECEL MP 943 from Aqualon, Wilmington, Del.; hydroxypropylcellulose, available as KLUCEL (LF, GF, MF, HF) from Aqualon; hydroxybutylmethylcellulose (3.5 wt-% hydroxybutyl and 30 wt-% methoxyl) from Scientific Polymer Products, Ontario, N.Y.; and hydroxyethylcelluloses, available under the trade designation NATROSOL from Aqualon. Xanthan gum, guar, locust bean gum, and other polysaccharides may also be suitable. These polymers may be produced from plant sources or can be produced through microbial cell culture. Polyvinyl alcohol (PVA) also may be suitable. For example, PVA made from polyvinyl acetate, which has been hydrolyzed to about 87%, is highly water soluble at room temperature.

The gel can also be thickened using suitable emulsifiers such as alkyl alcohols and polyethoxylated alkyl chain surfactants that effectively thicken the composition. Examples include the Polawax, Behenyl TMS, Crodaphos CES, Cosmowax, and Crothix systems from Croda Inc.

The gels are typically thickened to achieve a viscosity in excess of 500 cps, 1000 cps, 2000 cps, 3000 cps, 4000 cps, 5000 cps. Typically the viscosity in excess of 10,000 cps, more preferably greater than 25,000 cps and most preferably greater than 50,000 cps.

Compositions of the present invention may additionally employ adjunct components known in the art such as dyes, perfumes, fragrances, lubricants, thickening agents, stabilizers, skin penetration enhancers, preservatives, antioxidants, additional (cationic or non-ionic) antimicrobial, a flavoring agent (particularly for oral uses); providing the inclusion of such adjunct components does not detract from the combination of biguanide antimicrobial enhanced by the saturated or unsaturated (e.g. acetylene) diol surfactant.

Preferred compositions of the present invention exhibit good chemical stability. This can be especially a concern with compounds that may hydrolyze or undergo heat and/or light degradation such as chlorhexidine. The composition preferably retains an average of at least 97% of the antimicrobial component after aging for 4 weeks at 40° C. in a sealed container beyond the initial 5-day equilibration period at 23° C.

The composition described herein can reduce and eliminate Staphylococcus aureus and Pseudomonas aeruginosa. Those of ordinary skill in the art will readily determine when a composition provides antimicrobial activity using assay and bacterial screening methods well known in the art. For example, the compositions can be evaluated utilizing Antimicrobial Efficacy Test described in the Examples Section. Briefly, a bacterial suspension is introduced into a liquid composition. After a sufficient contact time, the sample containing the exposed bacteria is collected, placed in neutralizing broth, a sample is taken and diluted, and plated on growth medium. The plated sample is incubated at an appropriate temperature and humidity for twenty four to forty-eight hours and the number of viable bacterial colonies growing on the plate is counted. Once colonies have been counted the reduction in the number of bacteria caused by the test composition is readily determined. Bacterial reduction is generally reported as log₁₀ reduction determined by the difference between the log₁₀ of the initial inoculum count (i.e. the control) and the log₁₀ of the inoculum count after exposure.

Compositions were tested as described in the Examples Section for antimicrobial activity against Staphylococcus aureus (Gram positive, strain number 15981) and Pseudomonas aeruginosa (Gram negative, American Type Culture Collection (ATCC) strain number 15442 or 9027). Preferred compositions of the present invention also exhibit very rapid antimicrobial activity. In some embodiments, the compositions have an average of at least a 0.5, 1, 1.5 or 2 log reduction in test bacteria in 15 seconds, 30 seconds or 60 seconds. In other embodiments, the compositions have an average of at least a 2.5, 3, 3.5, 4, 4.5, 5 or 6 log reduction in test bacteria in 15 seconds, 30 seconds or 60 seconds.

In some embodiments, the improvement by inclusion of the saturated or unsaturated (e.g. acetylenic) diol surfactant is 50%, 100%, 200%, 300%, 400%, 500% or greater relative to the biguainde antimicrobial alone at the same concentration (difference in log₁₀ reduction divided by control)×100%. For example in one embodiment, 0.1wt-% of CHG alone provided a log10 reduction of 0.32; whereas when in combination with DYNOL, the log10 reduction was in excess of 6; i.e. a 20× improvement (2000%).

In other embodiments, the composition described herein can reduce and eliminate (e.g. Pseudomonas aeruginosa) biofilms using assay and bacterial screening methods well known in the art. For example, the compositions can be evaluated by use of a Minimum Biofilm Eradication Concentration device described in the Examples section. A biofilm is an aggregate of microorganisms that are adhered to each other on a surface through an extracellular polymeric matrix.

The compositions can also be utilized in provide effect topical antimicrobial activity and utilized in a method of treatment or prevention of a wide variety of affliction that are caused or aggravated by microorganisms on for example the skin, mucous membranes, and/or oral cavity surfaces, including teeth. In some embodiments, the composition may be used when there are no clinical indications of an affliction.

The compositions can be delivered from swabs, brush, woven cloth, sponges, foams, fibers, and non-woven and paper substrates (e.g., paper towels and wipes). In typical embodiments, a sufficient amount of the composition is applied to a surface and allowed to remain on the surface when applied in a dose, at a frequency, and in an amount sufficient to reduce or eliminate the microorganisms on the surface.

In another embodiment, a method of inhibiting biofilm formation on a surface of the oral cavity of a subject is described, comprising the steps of (1) providing a composition as described herein, and (2) applying the composition to a surface in the oral cavity of a subject. The surface in the oral cavity of a subject includes, for example, a buccal surface, a gingival surface, a tooth, a dental restoration, and bone. The composition may be applied to the oral cavity of a subject by, for example, immersing, inserting, rinsing, spraying, brushing, swabbing, or combinations thereof. Spraying the composition may provide the composition in the form of, for example, an aerosol or a fine mist. The subject may be a human, or the subject may be a non-human animal. Non-human animals include mammals such as canines and felines.

In another embodiment, a kit is described comprising (1) a composition as described herein, and (2) an application accessory. The application accessory can be a container, a sprayer, a brush, a swab, a tray, and combinations thereof. The application accessory can be any size. The kit may include more than one application accessory or more than one kind of application accessory (i.e., a sprayer and a brush). The kit may also include instructions for using the kit.

The compositions described herein are also useful for cleaning and disinfecting hard surfaces. Such compositions are largely aqueous, and are readily pourable and pumpable when packaged from a manually operable pump, such as a ‘trigger spray’ dispenser. In some embodiments, the cleaned and disinfected article comprises a hard surface and the dried composition described herein. The dried composition may also provide some residual sanitizing activity.

Hard surfaces include for example, glass, ceramic (for example, ceramic tile), cement, stone, porcelain, painted and/or clearcoat surfaces , appliances, plastic protective films which are backed with pressure-sensitive adhesives, metal (for example, architectural columns, plumbing fixtures), fiberglass, thermosetting polymers, sheet molding compound, thermoplastics (for example, polycarbonate, acrylics, polyolefins, polyurethanes, polyesters, polyamides, polyimides, phenolic resins, cellulose diacetate, cellulose triacetate, polystyrene, and styrene-acrylonitrile copolymers), and combinations thereof. Additional exemplary substrates include kitchen and bathroom hard surface such as bathtubs, toilets, sinks, faucets, mirrors, windows, and white boards.

Although care must still be taken to insure that the biguanide antimicrobial compound(s) are not inactivated by the inclusion of additional components, it is appreciated that cleaning and disinfecting compositions for hard surfaces, rather than skin, can include a vast array of additives that may not be suitable for skin contact uses.

The compositions can be applied to a surface of an article using conventional coating techniques, such as brush, bar, roll, wipe, curtain, rotogravure, spray, or dip coating techniques. One method is to apply the composition using any suitable method and, after allowing a portion of the liquid to evaporate.

The compositions are suitable for use in a consumer “spray and wipe” application as a cleaning composition. In such an application, the consumer generally applies an effective amount of the composition using the pump and within a few moments thereafter, wipes the treated area with a cloth, towel, or sponge, typically a disposable paper towel or sponge. Such application materials may be hydrophilic or hydrophobic in nature, preferably hydrophilic.

The composition described herein can also be applied to a hard surface by the use of a carrier substrate, as previously described. One example of a useful carrier substrate is a wet wipe. The wipe can be of a woven or non-woven nature. Fabric substrates can include non-woven or woven pouches, sponges including both closed cell and open celled sponges, including sponges formed from celluloses as well as other polymeric material, as well as in the form of abrasive or nonabrasive cleaning pads. Such fabrics are known commercially in this field and are often referred to as wipes. Such substrates can be resin bonded, hydroentangled, thermally bonded, meltblown, needlepunched, or any combination of the former. The carrier substrate useful with the present inventive compositions may also be a wipe which includes a film forming substrate such as a water soluble polymer. Such self-supporting film substrates may be sandwiched between layers of fabric substrates and heat sealed to form a useful substrate.

For antiseptic and cleaning uses, the compositions of the present invention are advantageously absorbed or disposed onto the carrier substrate, i.e., a wipe to form a saturated wipe. The wipe can then be sealed individually in a pouch which can then be opened when needed or a multitude of wipes can be placed in a container for use on an as needed basis. The container, when closed, sufficiently sealed to prevent evaporation of any components from the compositions. In use, a wipe is removed from the container and then wiped across an area in need of treatment; in case of difficult to treat stains the wipe may be re-wiped across the area in need of treatment, or a plurality of saturated wipes may also be used.

Objects and advantages of this disclosure are further illustrated by the following non-limiting examples, but the particular materials and amounts thereof recited in these examples, as well as other conditions and details, should not be construed to unduly limit this disclosure.

TABLE 1
MATERIALS

| Chemical | Source |
|---|---|
| DYNOL 604 SURFACTANT, 2,5,8,11- | Air Products and Chemicals |
| Tetramethyl-6-dodecyn-5,8-diol | Inc., Allentown, PA |
| ethoxylate, | |
| DYNOL 607 SURFACTANT | Air Products and Chemicals Inc. |
| SURFYNOL 104 SURFACTANT, | Air Products and Chemicals Inc. |
| 2,4,7,9-Tetramethyl-5-decyne-4,7- | |
| diol | |
| SURFYNOL 485 SURFACTANT | Air Products and Chemicals Inc. |
| Chlorhexidine gluconate (CHG) | MP Biomedicals, Santa Ana, CA |
| Polyhexamethylene biguanide | Lonza Chemicals, Allendale, NJ |
| (PHMB) (Cosmocil CQ) | |
| SURFYNOL AD01 SURFACTANT, | Air Products and Chemicals Inc. |
| 2,4,7,9-Tetramethyldecane-4,7- | |
| diol | |

### MICROBIOLOGICAL METHODS

Staphylococcus aureus strain 15981 (Valle J et al. Mol Microbiol. 2003 May; 48(4):1075-87) or Pseudomonas aeruginosa American Type Culture Collection strain number 9027 was grown to stationary phase in tryptic soy broth (Becton, Dickinson and Company, Franklin Lakes, N.J.) at 37° C. A volume of 2 mL of the stationary phase culture was centrifuged at 13000 RPM for 5 minutes in an Eppendorf Microfuge centrifuge in order to pellet the cells. The bacteria were then resuspended in 2 mL of sterile, deionized water, centrifuged again, and finally resuspended in 10 mL of sterile, deionized water. A volume of 180 μl of each antimicrobial formulation was dispensed into a polystyrene 96 well plate in triplicate. 20 μl of the bacterial suspension was added to each well containing antimicrobial formulations or control solutions for 15 to 60 seconds. After the incubation period, 20 μl was removed from each well and transferred to 180 μl of Dey/Englay (D/E) broth (Becton, Dickinson and Company, Franklin Lakes, NJ) in order to neutralize antimicrobial materials. The bacteria in D/E broth were serially diluted 10-fold in phosphate buffered saline and 100 μl aliquots of each dilution were transferred to 3M PETRIFILM AEROBIC COUNT PLATES (3M Co, St. Paul, Minn.). PETRIFILM plates were incubated for 24 hours at 37° C. and surviving colony forming units (CFUs) were counted. The average log (CFU/mL) of surviving bacteria was calculated. Samples were performed in triplicate, unless otherwise noted. Accordingly, for each example, surfactants were diluted in deonized water and chlorhexidine gluconate was added from a 20% stock solution when necessary.

### COMPARATIVE EXAMPLES: C1-C₃

### EXAMPLES: EX. 1-EX. 2

The addition of SURFYNOL 104 to 0.1% CHG enhanced the ability of the formulation to kill gram positive bacteria, S. aureus as shown in Table 2.

TABLE 2
Survival of S. aureus after 30 seconds and 60 seconds exposure
to 0.1% CHG with varying concentrations of SURFYNOL 104

| | 30 Seconds | Reduction | 60 Seconds | Reduction |
|---|---|---|---|---|
| | Ave. log | Relative to | Ave. log | Relative to |
| EXAMPLES | (CFU/mL) | Control | (CFU/mL) | Control |
| C1: 0.025% SURFYNOL 104 | 9.43 | None | 9.46 | None |
| C2: 0.0025% SURFYNOL 104 | 9.50 | None | 9.33 | 0.06 |
| C3: 0.1% CHG | 9.13 | 0.23 | 8.40 | 0.99 |
| EX. 1: 0.025% SURFYNOL 104 + | 8.28 | 1.08 | 6.90 | 2.49 |
| 0.1% CHG | | | | |
| EX. 2: 0.0025% SURFYNOL 104 + | 8.64 | 0.72 | 7.85 | 1.54 |
| 0.1% CHG | | | | |
| PBS (phosphate buffered saline) | 9.36 | | 9.39 | |
| control | | | | |

### COMPARATIVE EXAMPLE: C4

### EXAMPLES: EX. 3-EX. 6

The addition of DYNOL 604 or SURFYNOL 104 to 0.01% CHG enhanced the ability of the formulation to kill P. aeruginosa in an exposure time of 15 seconds as shown in Table 3.

TABLE 3
Survival of P. aeruginosa after 15 seconds exposure to 0.01%
CHG with varying concentrations of DYNOL 604 or SURFYNOL 104

| | | Reduction |
|---|---|---|
| | Average log | Relative to |
| EXAMPLES | (CFU/mL) | Control |
| C4: 0.01% CHG | 5.39 | 3.85 |
| EX. 3: 0.25% DYNOL 604 + 0.01% CHG | <3 | >6.24 |
| EX. 4 0.1% DYNOL 604 + 0.01% CHG | <3 | >6.24 |
| EX. 5 0.025% SURFYNOL 104 + 0.01% CHG | <3 | >6.24 |
| EX. 6 0.0025% SURFYNOL 104 + 0.01% | 5.58 | 3.66 |
| CHG | | |
| PBS control | 9.24 | |

Limit of detection was 3 log (CFU/mL)

### COMPARATIVE EXAMPLES: C5-C9

### EXAMPLES: EX. 7-EX. 10

The addition of DYNOL 604 or DYNOL 607 to 0.1% CHG enhanced the ability of CHG to kill S. aureus in a short period of time, as shown in Table 4. The surfactants alone at the same concentrations did not affect survival of the bacteria, indicating a synergistic effect of the combination of CHG and the nonionic surfactants.

TABLE 4
Survival of S. aureus after 30 seconds exposure to 0.1%
CHG with varying concentrations of DYNOL 604 or DYNOL 607

| | DYNOL 604 | Reduction | DYNOL 607 | Reduction |
|---|---|---|---|---|
| | Ave. log | Relative to | Ave. log | Relative to |
| EXAMPLES | (CFU/mL) | Control | (CFU/mL) | Control |
| C5: 0.1% DYNOL | 8.90 | 0.29 | 9.08 | 0.11 |
| C6: 0.25% DYNOL | 8.92 | 0.27 | 9.05 | 0.14 |
| C7: 0.75% DYNOL | 8.94 | 0.25 | 9.11 | 0.08 |
| C8: 1.0% DYNOL | 8.95 | 0.24 | 9.11 | 0.08 |
| C9: 0.1% CHG (no surfactant) | 8.87 | 0.32 | 8.87 | 0.32 |
| EX. 7: 0.1% DYNOL + 0.1% CHG | <3 | >6.19 | <3 | >6.19 |
| EX. 8 0.25% DYNOL + 0.1% CHG | <3 | >6.19 | 3.70 | 5.49 |
| EX. 9 0.75% DYNOL + 0.1% CHG | <3 | >6.19 | 5.29 | 3.90 |
| EX. 10 1.0% DYNOL + 0.1% CHG | <3 | >6.19 | 5.36 | 3.83 |
| PBS control | 9.19 | | 9.19 | |

Limit of detection was 3 Log (CFU/mL)

### COMPARATIVE EXAMPLES: C10-C13

### EXAMPLES: EX. 11-EX. 14

The addition of ethanol to solutions of CHG and the nonionic surfactants did not disrupt the synergistic effect of their combination as shown in Table 5.

TABLE 5
Survival of S. aureus after 30 seconds exposure
to 0.1% CHG plus either 0.1% DYNOL 607 or 0.1%
SURFONYL 485 in the presence of 5% ethanol.

| | | Reduction |
|---|---|---|
| | Ave. log | Relative to |
| EXAMPLES | (CFU/mL) | Control |
| C10: 0.1% CHG | 7.82 | 0.62 |
| C11: 0.1% CHG, 5% EtOH | 7.17 | 1.27 |
| C12: 0.1% SURFYNOL 485 | 8.50 | None |
| EX. 11: 0.1% SURFYNOL 485, 0.1% CHG | 7.44 | 1.00 |
| EX. 12: 0.1% SURFYNOL 485, 0.1% CHG, 5% | 7.02 | 1.42 |
| EtOH | | |
| C13: 0.1% DYNOL 607 | 8.51 | None |
| EX. 13: 0.1% DYNOL 607, 0.1% CHG | 4.21 | 4.23 |
| EX. 14: 0.1% DYNOL 607, 0.1% CHG, 5% | 3.81 | 4.63 |
| EtOH | | |
| PBS control | 8.44 | |

### COMPARATIVE EXAMPLES: C14-C18

### EXAMPLES: EX. 15-EX. 18

SURFYNOL 104 was combined with polyhexamethylene biguanide (PHMB) and the resulting combinations were tested for their ability to kill bacteria growing in biofilms, communities of bacteria that attach to surfaces. Bacterial biofilms were grown on a device that generates 96 biofilms in a 96-well plate format. The biofilms are then exposed to various liquid formulations in a standard 96-well plate. It was observed that SURFYNOL 104 enhanced the ability of PHMB to kill Pseudomonas aeruginosa in biofilms, as shown in Table 6.

Pseudomonas aeruginosa biofilms were grown in a Minimum Biofilm Eradication Concentration (MBEC) device (Innovotech, Edmonton, AB, Canada). The MBEC device consists of standard polystyrene, 96-well plate and a polystyrene lid with attached pegs that protrude from the lid so that they dip into the wells of the 96-well plate. Cultures of American Type Culture Collection (ATCC), Pseudomonas aeruginosa ATCC #15442 were grown for 18 hours in trypticase soy broth (TSB, Becton Dickinson, and Company, Franklin Lakes, N.J.) at 37° C. The 18 hour culture was diluted 1:10,000 in fresh TSB and 150 μl of diluted culture was transferred to each well of the MBEC device and the lid of the MBEC plate was placed on the device so that the pegs of the lid dipped into the bacterial suspension of the plate. The plate was then incubated for 24 hours at 37° C.

After 24 hours, the lid of the MBEC device was transferred to a 96-well plate containing 200 μl of sterile water in each well to remove any loosely attached bacteria from the pegs of the MBEC device. The biofilm-covered pegs were then transferred to a 96-well plate containing 200 μl of the exposure solutions in each well. The biofilms were exposed to the antimicrobial or control formulations for 6 minutes after which they were transferred to a new 96-well plate containing 210 μl of Dey/Englay neutralization broth (Becton, Dickinson, and Company, Franklin Lakes, N.J.) in each well. The bacteria were recovered from the device by sonication on a floating steel tray of a sonicating water bath for 30 minutes (Branson Ultrasonics, Danbury, Conn.). After sonication, the bacterial suspensions were serially diluted 10-fold in a 96-well plate and plated on 3M PETRIFILM AEROBIC COUNT PLATES (3M Co, St. Paul, Minn.). The plates were incubated for 48 hours at 37° C. The colony forming units (CFUs) were enumerated and the average (triplicate samples) log(CFU/peg) surviving in each exposure condition was calculated.

TABLE 6
Survival of P. aeruginosa biofilms in MBEC Device
after 6 minute exposure to various concentrations
of DYNOL 604 or SURFYNOL 104, +0.1% PHMB

| | | Reduction |
|---|---|---|
| | Ave. log | Relative to |
| EXAMPLES | (CFU/mL) | Control |
| Water control | 7.51 | |
| C14: 0.1% PHMB | 5.52 | 1.99 |
| C15: 0.1% DYNOL 604 | 6.98 | 0.53 |
| C16: 0.25% DYNOL 604 | 7.12 | 0.39 |
| EX. 15: 0.1% DYNOL 604 + 0.1% PHMB | 5.99 | 1.52 |
| EX. 16 0.25% DYNOL 604 + 0.1% PHMB | 5.85 | 1.66 |
| C17: 0.025% SURFYNOL 104 | 7.62 | None |
| C18: 0.0025% SURFYNOL 104 | 7.60 | None |
| EX. 17 0.025% SURFYNOL 104 + 0.1% PHMB | 4.07 | 3.44 |
| EX. 18 0.0025% SURFYNOL 104 + 0.1% | 4.69 | 2.82 |
| PHMB | | |

### COMPARATIVE EXAMPLES: C19-C22

### EXAMPLES: EX. 19-EX. 20

The method of EX. 1-2 was used with the exception that SURFYNOL AD01 was used instead of SURFYNOL 104 in the formulations. The addition of SURFYNOL AD01 to 0.1% CHG enhanced the ability of the formulation to kill S. aureus in an exposure time of 30 seconds as shown in Table 7.

TABLE 7
Survival of S. aureus after 30 seconds exposure to 0.1%
CHG plus either 0.05% SURFYNOL AD01 or 0.1% SURFYNOL AD01.

| | | Reduction |
|---|---|---|
| | Ave. log | Relative to |
| EXAMPLES | (CFU/mL) | Control |
| C19: 0.1% CHG | 7.55 | 0.65 |
| C20: 0.05% SURFYNOL AD01 | 8.09 | 0.11 |
| C21: 0.1% SURFYNOL AD01 | 8.25 | None |
| C22: 1% SURFYNOL AD01 | 8.32 | None |
| EX. 19: 0.05% SURFYNOL AD01, 0.1% CHG | 6.75 | 1.45 |
| EX. 20: 0.1% SURFYNOL AD01, 0.1% CHG | 6.55 | 1.65 |
| PBS control | 8.20 | |

What is claimed is:

1. A method for cleaning and disinfecting comprising
providing an antimicrobial composition comprising
at least one biguanide antimicrobial compound;
at least one hydrocarbon surfactant enhancer having the formula: 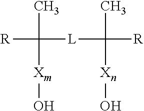
wherein L is a covalent bond or a saturated or unsaturated C₁-C₂ moiety,
R is independently a straight chained or branched C₁-C₁₂ alkyl moiety,
X is independently a C₂-C₄ alkoxy,
m+n ranges from 0 to about 50; and
an aqueous carrier comprising at least 50 wt. % water; and
applying the antimicrobial composition to a hard surface.

2. The method of claim 1 wherein the hard surface is a kitchen or bathroom surface.

3. The method of claim 1 wherein the aqueous carrier further comprises an alkyl alcohol, ether glycol, or combination thereof.

4. The method of claim 1 wherein the antimicrobial composition further comprises a thickener.

5. The method of claim 1 wherein the biguanide antimicrobial compound is chlorhexidine gluconate (CHG) or polyhexamethylene biguanide (PHMB).

6. The method of claim 1 wherein the biguanide antimicrobial compound is present at a concentration less than 5 wt-%.

7. The method of claim 1 wherein the biguanide antimicrobial compound(s) and surfactants(s) are present at a weight ratio ranging from 100:1 to 1:10.

8. The method of claim 1 wherein the hydrocarbon surfactant is present in an amount such that the antimicrobial activity of the biguanide antimicrobial compound is improved.

9. The method of claim 1 wherein the antimicrobial composition is absorbed or disposed onto a wipe.

10. The method of claim 1 wherein the antimicrobial composition is applied to the hard surface by spraying the antimicrobial composition.

11. The method of claim 1 further comprising and at least partially removing the aqueous or hydrophilic liquid from the hard surface by wiping with a cloth, paper towel, or sponge.

12. An article comprising a hard surface and the dried antimicrobial composition according to claim 1.

13. A method for reducing biofilm comprising
providing an antimicrobial composition comprising
at least one biguanide antimicrobial compound;
at least one hydrocarbon surfactant enhancer having the formula: 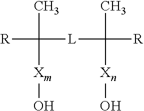
wherein L is a covalent bond or a saturated or unsaturated C₁-C₂ moiety,
R is independently a straight chained or branched C₁-C₁₂ alkyl moiety,
X is independently a C₂-C₄ alkoxy,
m+n ranges from 0 to about 50; and
an aqueous carrier comprising at least 50 wt. % water; and
applying the antimicrobial composition to mammalian skin, mucosal tissue, or an oral cavity.

14. The method of claim 13 wherein the aqueous carrier further comprises an alkyl alcohol, ether glycol, or combination thereof.

15. The method of claim 13 wherein the biguanide antimicrobial compound is chlorhexidine gluconate (CHG) or polyhexamethylene biguanide (PHMB).

16. The method of claim 13 wherein the biguanide antimicrobial compound is present at a concentration less than 5 wt-%.

17. The method of claim 13 wherein the biguanide antimicrobial compound(s) and surfactants(s) are present at a weight ratio ranging from 100:1 to 1:10.

18. The method of claim 13 wherein the hydrocarbon surfactant is present in an amount such that the antimicrobial activity of the biguanide antimicrobial compound is improved.
